(19) United States
(12) Patent Application Publication
(10) Pub. No.: US 2016/0051716 A1
(43) Pub. Date: Feb. 25, 2016

(54) THERMALLY EFFICIENT PORTABLE VAPORIZER HEATING ASSEMBLY

(71) Applicant: Vaporfection International, Inc., Deerfield Beach, FL (US)

(72) Inventor: Franklin R. Wheelock, Dallas, GA (US)

(21) Appl. No.: 14/462,799

(22) Filed: Aug. 19, 2014

(51) Int. Cl. A61L 9/03; A61M 11/04; F22B 1/28

(52) U.S. Cl. CPC A61L 9/03; F22B 1/28; A61M 11/042

(57) ABSTRACT

A portable hand-held vaporizer assembly having a glass body defining an airflow passage is disclosed. A heating element is thermally coupled to the glass body and a material placement zone is downstream from the airflow passage. Furthermore, a thermally conductive layer covers at least a portion of an outer surface of the glass body and a thermally reflective substance is included with walls encapsulating the glass body and the heating element.

### FIELD OF THE INVENTION

The present invention relates generally to a vaporizer for inhalation of active ingredients within a plant material, and more particularly relates to a portable, concealable, hand-held, vaporizer utilizing convection heating and a spiral airflow chamber, including a thermally conductive layer and a thermally reflective layer to reduce thermal inefficiencies.

### BACKGROUND OF THE INVENTION

A vaporizer is a device used to extract the active ingredients of plant material, e.g., tobacco, or other herbs or blends, for inhalation by a human. Vaporization involves heating a material so that its active compounds boil off into a vapor. As opposed to smoking, i.e., burning, vaporization avoids the production of irritating, toxic, and carcinogenic by-products. In fact, no combustion occurs, so no smoke or taste of smoke is present. Studies show that vapor contains substantially zero particulate matter or tar, and, in comparison to smoking, significantly lower concentrations of noxious gases such as carbon monoxide. It has also been shown that, in comparison to other drug delivery methods, such as ingestion, vaporization has a more rapid onset of pharmacological effect, direct delivery into the bloodstream (via the lungs), and more precise titration such that the desired level is reached and not exceeded, enabling consistent and appropriate dosage.

Generally, those vaporizers utilizing convection-based heating methods employ the use of a heating element by which air passes through, or is in contact with, such that the temperature of the air is heated sufficiently to extract an herb's natural ingredients. To remove an herb's active ingredients the effective vapor temperature varies depending on the type of supplied herb, but generally ranges from 350 to 680 degrees Fahrenheit. After the active ingredients from the herb are boiled off into a vapor, it generally is too hot to be comfortably and/or safely inhaled by a human being. As such, many vaporizes utilize hoses (also called “whips”), elongated chambers, and large heat sinks to reduce the temperature of the vapor so it is safe and comfortable for human inhalation. For most known vaporizers, the vaporization process requires an assembly or casing that is cumbersome and not easily portable. Those assemblies that are portable are too large to conceal within a standard-sized pant pocket and do not allow the device to be effectively and conveniently transported. Most of these vaporizes also require the unit to be plugged in to an electric outlet, which is inconvenient for those persons without access to electricity.

To effectively reduce the temperature of the vapor so it can be consumed, many hand-held vaporizers utilize an elongated chamber that reduces the vapor to a desired temperature. This elongated chamber commonly creates an assembly that is in the general shape of a flashlight with a nozzle-type mouthpiece at the end. There are numerous disadvantages associated with the elongated shape of the vaporizer. With the mouthpiece coupled to the end of the device it becomes readily identifiable as a smoking-type apparatus, which many users find undesirable. Few, if any, of those hand-held vaporizers easily conceal the mouth piece, without removal into multiple components, which can be easily misplaced and/or dropped. As most mouth pieces are not easily concealable, and because they generally have at least one opening disposed to the outside environment to inhale the vapor, the device also becomes susceptible to liquids and debris from entering, which may contaminate the device's functionality. Those vaporizers that are shaped in an elongated fashion also typically require the vaporizer to be subject to the time-intensive task of disassembling it into multiple components in order for the user to insert the herbs. As such, few vaporizers allow a user to insert and remove herbs into the vapor chamber quickly and efficiently without at least partially disassembling the device.

To allow the vapor temperature to reduce to comfortable and safe ranges, many known vaporizers have their heating element at a location 4-6 inches away from the mouthpiece or inhaling area. In such configurations, which are in-line with most known vaporizers that are elongated, the vapor is given more time to dissipate the heat. This, however, negatively creates an inconsistent temperature at the mouthpiece that is dependent on the inhaling rate of the user. For example, as a user inhales faster, the air from the outside environment passes more quickly past the heating element which in turn reduces the temperature at the mouthpiece. A slower inhale rate produces a higher temperature at the mouthpiece because the incoming air is in contact with the heating element longer. This can lead to a less effective release of the active ingredients in the herbs, should the user inhale very fast. Furthermore, as the temperature generally varies, depending on the inhaling rate, many hand-held vaporizers require a user to inconveniently adjust their breathing rate to produce the desired temperature at the mouth piece. As the temperature generally varies, most known portable hand-held vaporizers do not allow the user to have an optimized air flow, a safe vapor temperature at the mouth piece, and a highly potent vapor containing the herb's active ingredients.

To compensate for the sporadic temperature at the mouthpiece and to form a more compact body of the vaporizer, many known vaporizers utilize conduction-based, as opposed to convection-based, heating, i.e. direct contact of the herb with a heated material. These known vaporizers overcome sporadic vapor temperatures by placing the heating element closer to the mouthpiece. These vaporizers are able to accomplish this as there is no fluid that is required to be heated. Conduction vaporizers have their own set of problems, however, and are generally considered inferior to convection-based heated vaporizers. Convection heating is more efficient as the heated fluid, “air,” is in contact with more surface area of the herb. This in turn provides a more potent vapor and does not require the user to adjust the herbs. Convection-based heating vaporizers further allow a user to control the heating element more effectively than those conventional conduction-based heated vaporizers. This also allows for a generally more potent vapor, as the user increases the chances that all of the herb's active ingredients are boiled off into the vapor. As such, few, if any, known vaporizers are able to utilize convection heating into a compact discrete design that creates safe and comfortable vapor temperatures.

Prior-art vaporizers use a heating element, typically a metallic heating element, that adds an unpleasant taste to the air stream flowing through it. This unpleasant, contaminated, metallic taste takes away from the enjoyment, i.e. the user experience, of the vaporizing process. In addition, prior art vaporizers are made with a heating element inside an air-flow conduit where the heating element rests against a portion of the interior of the conduit, thereby preventing air from flowing around certain portions of the heating element, resulting in non-optimized airflow and/or heat exchange.

Many vaporizers are not thermally efficient, which is particularly troublesome with portable vaporizers that require high energy efficiency for optimal battery power life. For example, some prior-art vaporizers utilize materials, such as metal components, that absorb a large amount of heat, thus, requiring the expenditure of additional energy to generate optimum heat levels. Portable vaporizers, which are smaller, require optimal space management, as well as, thermal shielding. For example, certain portions of the portable vaporizer, such as electronic components, do not operate, or operate at lesser efficiencies, when exposed to high temperatures from the heating element.

Additionally, selecting proper materials and configurations for a portable vaporizer can be challenging, particularly where size considerations, power considerations, and user experience considerations are in tension with one another. For example, using an airflow chamber constructed of low thermally conductive materials, such as glass, is certainly an unexpected choice because, at first glance, heating air flowing through a low-conductive material would require more energy to heat than a highly-conductive material. On the other hand, a non-metallic material, such as glass, would not contaminate the airflow, as would a highly-conductive metallic material, thereby enhancing the user experience. Accordingly, portable vaporizers are needed that optimize both thermal energy management and space management, while providing an enjoyable user experience with clean, non-contaminated air.

Therefore, a need exists to overcome the problems with the prior art as discussed above.

### SUMMARY OF THE INVENTION

The invention provides a portable hand-held vaporizer assembly that overcomes the hereinafore-mentioned disadvantages of the heretofore-known devices and methods of this general type and provides a device that utilizes convection-based heating, using a glass airflow passage coated with a boron nitride layer to reduce a thermal inertia of glass. The invention also provides a portable vaporizer where the glass airflow passage is encompassed within walls impregnated with a thermally reflective material, such as copper-nickel, to reflect radiated heat to remain within the cavity.

With the foregoing and other objects in view, there is provided, in accordance with the invention, a vaporizer heating assembly having a glass body defining an airflow passage, a heating element thermally coupled to the glass body, a material placement zone downstream from the airflow passage, and a thermally conductive layer covering at least a portion of an outer surface of the glass body.

In accordance with one embodiment of the present invention, the thermally conductive layer comprises boron.

In accordance with another feature of the present invention, the thermally conductive layer comprises a boron nitride powder and an adhesive.

In accordance with a further feature of the present invention, the thermally conductive layer covers less than about one-third of the outer surface of the glass body.

In accordance with yet a further feature of the present invention, the glass body includes at least one spiral winding between a distal winding end and a proximal winding end, opposite the distal winding end, and the thermally conductive layer covers the proximal winding end and not the distal winding end.

In accordance with another feature of the present invention, the glass body includes a spiral glass body defining a spiral airflow passage, and a container fluidly coupled to the spiral airflow passage, the container including the material placement zone; and the thermally conductive layer covers a portion of an outer surface of the spiral glass body and a portion of the container.

In accordance with another feature of the present invention, a body including a plurality of walls defining a cavity within the body, the glass body and the heating element disposed within the cavity, and the plurality of walls including a thermally reflective substance operably configured to reflect infrared emissions within the cavity.

In accordance with another embodiment of the present invention, there is provided a vaporizer heating assembly having an airflow passage thermally coupled to a heating element, a material placement zone downstream from the airflow passage, and a body including a plurality of walls defining a cavity within the body, the airflow passage and the heating element disposed within the cavity, and at least one of the plurality of walls including a thermally reflective material.

In accordance with another feature of the present invention, the thermally reflective material comprises copper-nickel.

In accordance with yet another feature of the present invention, the plurality of walls is comprised of a polymer material impregnated with copper-nickel.

In accordance with a further feature of the present invention, the thermally reflective material is operably configured to reflect infrared emissions within the cavity.

In accordance with another feature of the present invention, the airflow passage is defined by a glass body.

In accordance with another feature of the present invention, the airflow passage is formed as a spiral airflow passage defined by a spiral glass body.

In accordance with yet another feature of the present invention, a thermally conductive layer covers at least a portion of an outer surface of a glass body defining the airflow passage.

In accordance with the present invention, a method of improving a thermal efficiency of a vaporizer heating assembly having a glass airflow passage is disclosed. The method includes providing a vaporizer heating assembly having a glass body defining an airflow passage, a heating element thermally coupled to the glass body, and a material placement zone downstream from the airflow passage. A thermally conductive layer is applied to at least a portion of an outer surface of the glass body.

In accordance with yet another feature, an embodiment of the present invention includes applying a boron nitride layer to at least a portion of an outer surface of the glass body.

In accordance with yet another feature, an embodiment of the present invention also includes mixing a boron nitride powder with an adhesive to form a paste-like substance; and applying the paste-like substance to at least a portion of an outer surface of the glass body.

In accordance with a further feature of the present invention, an embodiment includes providing the glass body and the heating element within a cavity defined by a plurality of walls, and including a thermally reflective substance within the plurality of walls to reflect infrared emissions within the cavity.

In accordance with yet another feature of the present invention, an embodiment includes providing the glass body and the heating element within a cavity defined by a plurality of walls; and including copper-nickel within the plurality of walls to reflect infrared emissions within the cavity.

In accordance with another feature of the present invention, an embodiment includes providing the glass body and the heating element within a cavity defined by a plurality of walls; and impregnating at least one of the plurality of walls with copper-nickel.

Although the invention is illustrated and described herein as embodied in a vaporizer heating assembly, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the heating element. The term “heating element,” as used herein, indicates a structure that actively produces heat and are directly physically coupled to such structures, as opposed to nearby structures that receive heat via convection and possibly transfer heat to other areas. The term “downstream,” as used herein indicates a location along a path of flow that is further down the path of flow and occurs after a reference point in that path of flow.

The terms “program,” “application,” “software application,” and the like as used herein, are defined as a sequence of instructions designed for execution on a computer system. A “program,” “computer program,” “application,” or “software application” may include a subroutine, a function, a procedure, an object method, an object implementation, an executable application, an applet, a servlet, a source code, an object code, a shared library/dynamic load library, and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a sectional front elevational view of a portable hand-held vaporizer heating assembly in accordance with an embodiment of the present invention (where a heating element of the assembly is not illustrated for clarity);

FIG. 2 is a sectional front perspective view of the portable hand-held vaporizer heating assembly of FIG. 1, in accordance with an embodiment of the present invention (where the heating element of the assembly is not illustrated for clarity);

FIG. 3 is an enlarged perspective view of a spiral airflow chamber surrounding a heating element of the portable hand-held vaporizer heating assembly of FIG. 1, illustrating thermal reflection of infrared emissions from the heat engine;

FIG. 4 is an enlarged side elevational view of the spiral airflow chamber and the heating element of the portable hand-held vaporizer heating assembly of FIG. 1;

FIG. 5 is an exploded perspective view of the portable hand-held vaporizer heating assembly of FIG. 1, illustrating a selectively removable herbal container and a selectively removable mouth piece in accordance with an embodiment of the present invention;

FIG. 6 is an enlarged perspective view of the selectively removable mouth piece of the portable hand-held vaporizer heating assembly of FIG. 1;

FIG. 7 is an assembled perspective view of the portable hand-held vaporizer heating assembly of FIG. 1, illustrating insertion of the herbal container and mouth piece with a body of the portable hand-held vaporizer heating assembly and showing the cover in an open position;

FIG. 8 is a perspective view of the portable hand-held vaporizer heating assembly of FIG. 1, illustrating the cover in a closed position;

FIG. 9 is a perspective view of an alternative embodiment of a spiral airflow chamber in accordance with the present invention;

FIG. 10 is a front elevational view of the spiral airflow chamber, originally introduced in FIG. 9; and

FIG. 11 is a flow chart of a method of improving thermal efficiency of a portable hand-held vaporizer heating assembly in accordance with the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient portable hand-held vaporizer assembly that provides a glass spiral airflow chamber coated with a thermally conductive boron nitride layer to reduce a thermal inertia of the glass spiral airflow chamber. In addition, embodiments of the invention provide for impregnating the wall surrounding the airflow chamber with a thermally reflective copper-nickel substance to reflect heat radiating from the heat engine, thereby conserving thermal energy.

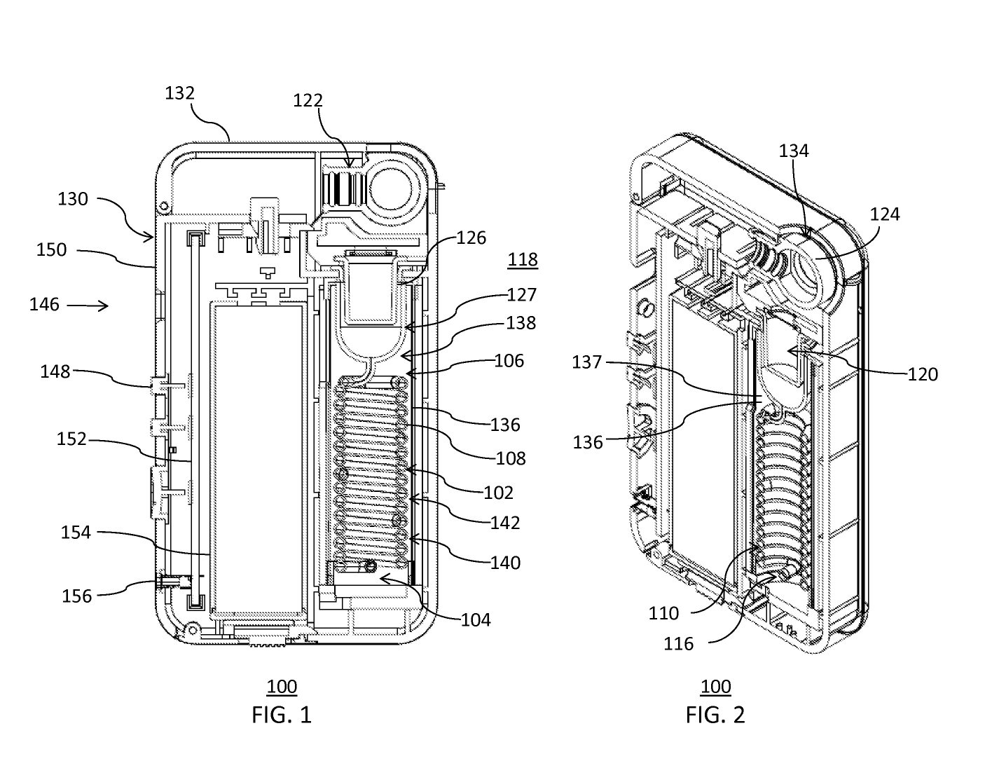

Referring now to FIGS. 1-4, one embodiment of the present invention is shown in a variety of views. The figures of the instant application show several advantageous features of the present invention, but, as will be described below, the invention can be provided in several shapes, sizes, combinations of features and components, and varying numbers and functions of the components.

FIGS. 1-4 illustrate an embodiment of a portable hand-held vaporizer heating assembly 100 in accordance with the present invention. The portable hand-held vaporizer heating assembly 100 includes an airflow chamber 102 having a distal end 104 and a proximal end 106, and a spiral glass body 108 coupling the distal end 104 to the proximal end 106. The spiral glass body 108 defines a spiral airflow passage 110 and continuously winds about an area 112. In one embodiment, the spiral glass body 108 can be formed as a tubular body, where a cross-section of the tubular body has a diameter defining a diameter of the spiral airflow passage 110. In another embodiment, the tubular body is not comprised of glass. In yet another embodiment, the area 112 can be seen as a space defined by and disposed within a series of closely compacted circular windings of the spiral glass body 108. In one embodiment, the windings of the spiral glass body 108 are concentric with one another and have substantially the same diameter with one another. In an alternative embodiment, the windings are substantially adjacent to one another, or touching, in order to maximize a length of the spiral airflow passage 110 that is thermally coupled to and heated by a heating element 114 of the assembly 100. The spiral configuration of the present invention results in efficient space management and efficient thermal distribution. As used herein, the term “spiral” is defined as a curve or a bend on a plane that winds around a fixed point at a continuously increasing or decreasing distance from the fixed point.

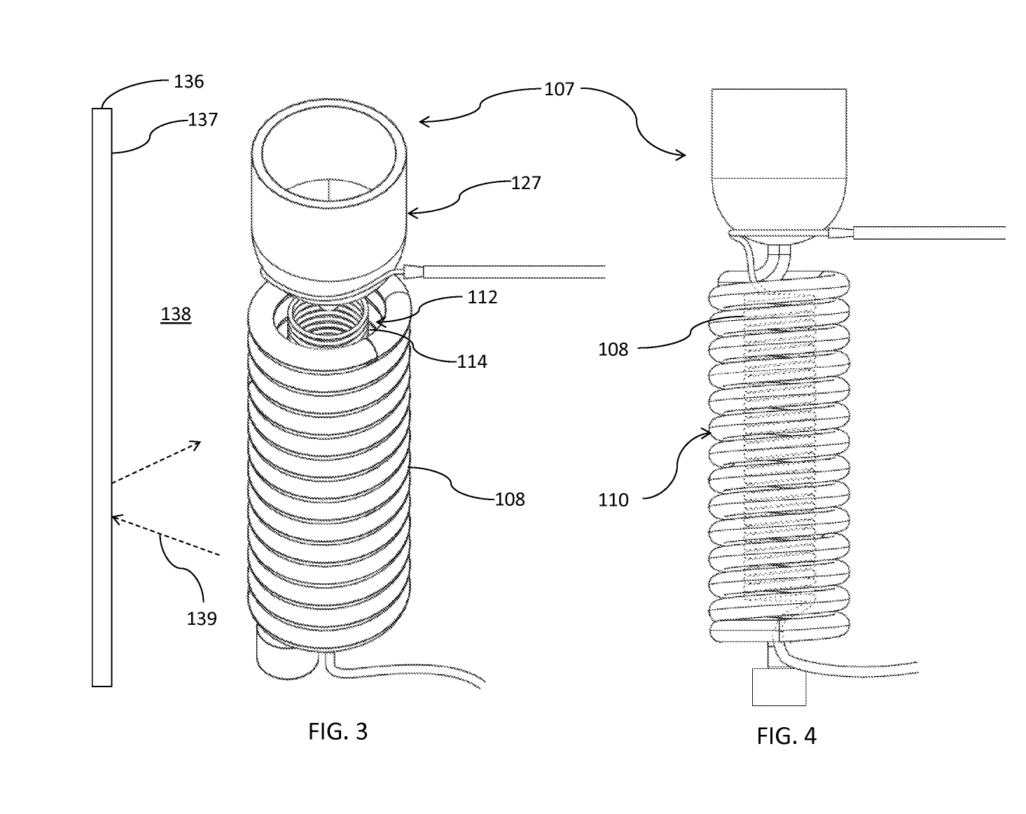

FIGS. 3-4 illustrate a heating assembly 107, which includes the spiral glass body 108 and the heating element 114. The entire heating assembly 107, or one or more portions thereof, e.g. the heating element 114, may also be referred to as the “heat engine” or “heating engine” as it is this component that thermally charges the incoming outside ambient air to a proper vaporizing temperature. The term “thermally coupled” is defined as having a first and second object or matter in relative proximity such that heat is effectively exchanged from the first object to the referenced second object or matter. In one embodiment, the heating element 114 is a tungsten-based metallic alloy in the form of a coil that is disposed at least partially within the airflow passage 110. In other embodiments, the heating element 114 is made from nickel-chrome, other types of metals, or metal-based composites that have a general low thermal resistivity and are generally safe to pass air through for human consumption. In further embodiments, the heating element 114 may be in the form of a plate or other shape, and may be located within a piece of glass or in close proximity to the airflow passage 110, but yet still able to effectively transfer heat.

Referring now again to FIGS. 1-4, the distal end 104 of the airflow chamber 102 defines a distal opening 116 that places the spiral airflow passage 110 in fluid communication with an outside environment 118. As defined herein, the term “outside environment” is defined as an environment outside an external housing of the portable hand-held vaporizer heating assembly 100. The distal opening 116 provides the assembly 100 with a steady stream of fluid, i.e. air, used for the convection heating process, which is more effective and efficient than other heating methods used by prior-art vaporizers. The heating element 114 is thermally coupled to the spiral airflow passage 110 and the spiral glass body 108, without contacting airflow in the spiral airflow passage 110. The heating element 114 is disposed within the area 112 about which the spiral glass body 108 continuously winds. As used herein, the term “winding” is intended to indicate wrapping around a center point or another object once or repeatedly. The heating element 114 can be in the form of a metallic wire or other conductive material, operably configured to radiate heat therefrom. In one embodiment, the heating element 114 is spiral-shaped. In another embodiment, the heating element 114 is substantially concentric with the spiral glass body 108. Advantageously, the heating element 114 is not within the airflow path of the spiral airflow passage 110 so that air flowing through the spiral airflow passage 110 is not contaminated with materials from the convection-based heating element 114, resulting in a metallic taste. A glass-on-glass airflow passage provides a long time of high-temperature operation without producing any additives or contaminants to the stream of air flowing therethrough, a problem plaguing the prior art, which uses materials such as ceramic. Moreover, glass is generally not known to be a highly thermally conductive material; therefore efficient thermal distribution is desired. Accordingly, providing the glass airflow passage in a spiral configuration, in accordance with the present invention, advantageously increases thermal distribution efficiency, in addition to, energy and space management efficiency within the portable hand-held vaporizer heating assembly 100. In a further embodiment, the heating element 114 is substantially adjacent to, or touching, an external surface of the spiral glass body 108 in order to further optimize space and thermal efficiency.

A material placement zone 120 is included in the assembly 100, downstream from the heating element 114 and downstream from the spiral airflow passage 110. A material to be placed within the material placement zone 120 can be, for example, an herb or other material intended to be heated by the portable hand-held vaporizer heating assembly 100. The material placement zone 120 is in fluid communication with the spiral airflow passage 110. A receptacle 126 defines the material placement zone 120. The receptacle 126 may be in the form of a cup, a bowl, a tray, or other container body operably configured to hold an herb in a cavity or on a support surface thereof, for being heated by heated air flowing through the spiral airflow passage 110. In one embodiment, the receptacle 126 is selectively removable from the assembly 100, as best illustrated in FIG. 18. In another embodiment, the assembly 100 includes a multi-receptacle carrier sized and configured to fit substantially within a small bag, or a pouch. The multi-receptacle carrier can include, for example, four receptacles 126, which can be pre-loaded by a user with herbs. In yet another embodiment, the receptacle 126 is comprised of glass. The receptacle 126 is preferably not comprised of a metal or other highly thermally conductive material, because the metal will absorb large amounts of heat, resulting in thermal inefficiency and requiring additional power in order to maintain heat levels at the proper vaporizing temperature. As with the receptacle 126, other components of the assembly 100 are preferably comprised of a material that is not highly thermally conductive for purposes of thermal and power efficiency. Preferably, in accordance with the present invention, air flowing through the spiral airflow passage 110 from the distal opening 116 to the material placement zone 120 only contacts glass or a material substantially comprised of glass. As used herein, the term “contact” is intended to indicate a direct, physical touch. In a further embodiment, air flowing through the spiral airflow passage 110 from the distal opening 116 to the material placement zone 120 does not contact at least one of a metal material and a plastic material. Plastic materials may deteriorate at certain high temperatures resulting in contamination of air flowing through the spiral airflow passage 110. In yet a further embodiment, air flowing through the spiral airflow passage 110 from the distal opening 116 to the material placement zone 120 does not contact the heating element 114.

The receptacle 126 may be supported by a container 127 fluidly coupled to the proximal end 106 of the spiral airflow passage 110. The container 127 includes the material placement zone 120 and can provide a support surface for the receptacle 126 that receives the herbal product therein. The container 127 can be in the form of a cup, a bowl, a tray, or other container body operably configured to hold the receptacle 126 within a cavity or on a support surface thereof, for being heated by heated air flowing through the spiral airflow passage 110. In one embodiment, the container 127 is made substantially of glass.

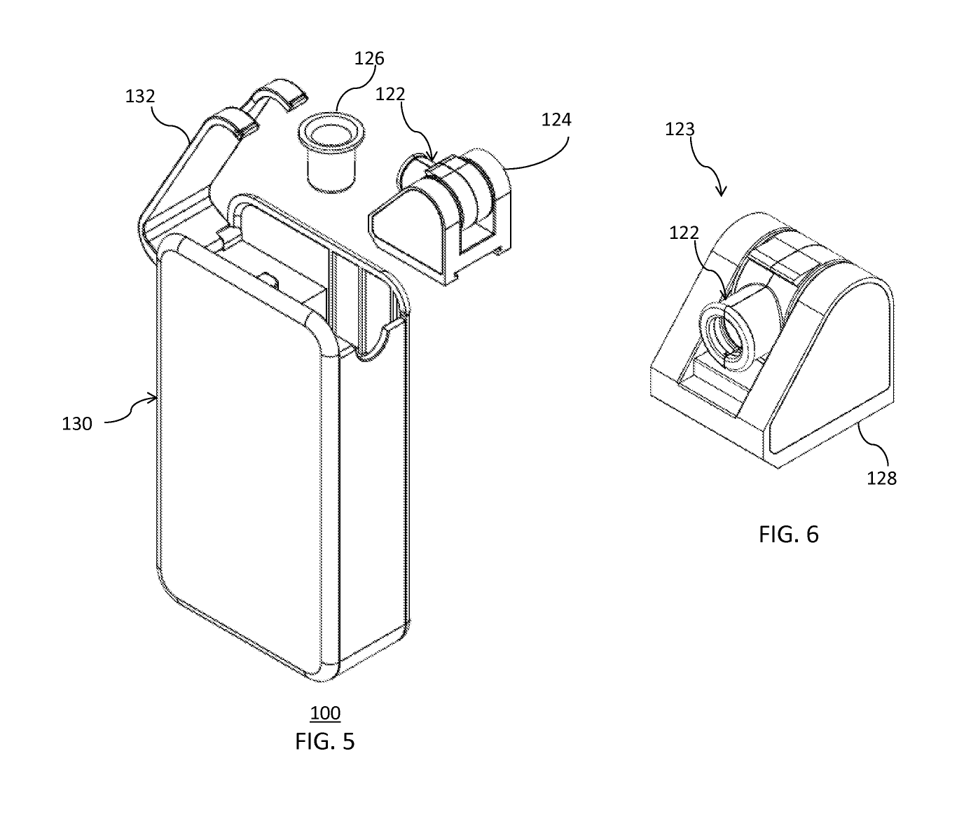

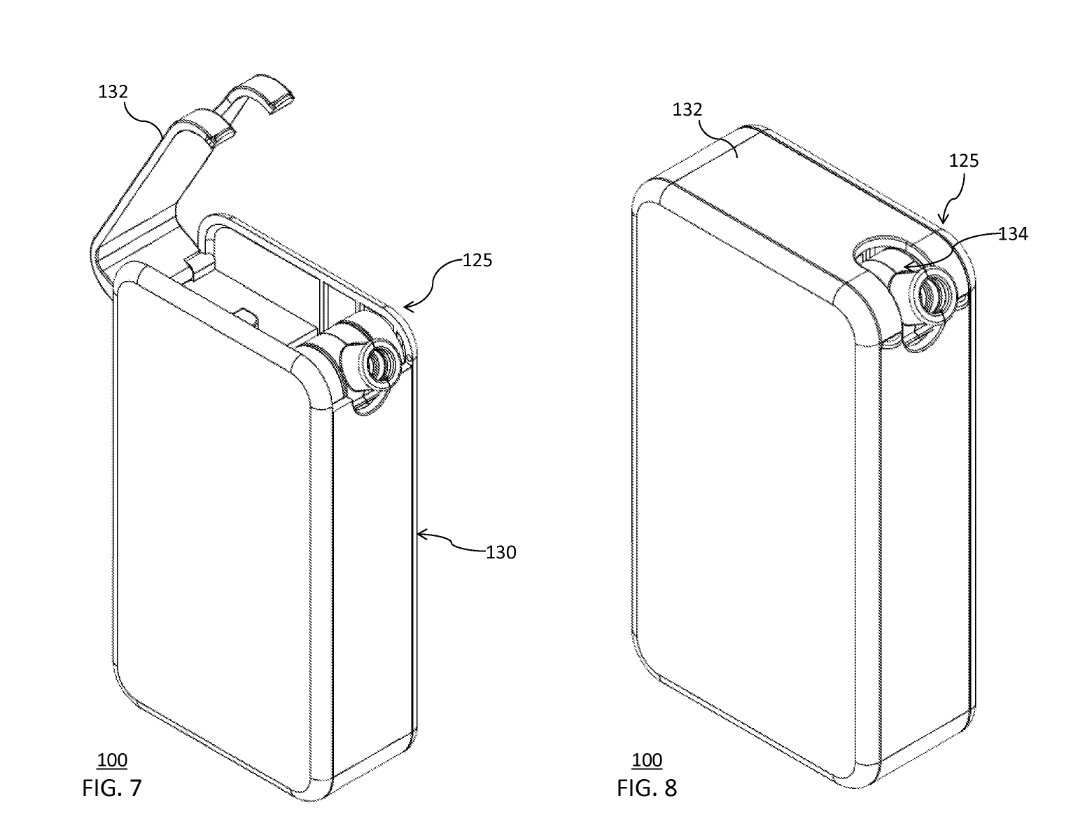

A conduit 122 places the spiral airflow passage 110 in fluid communication with the outside environment 118. In one embodiment, the conduit 122 is coupled to a selectively removable mouth piece 124, as best illustrated in FIGS. 5-6. In one embodiment, the conduit 122 is made substantially of glass. In an alternative embodiment, the conduit 122 is thermally conductive, or has the ability to transfer heat across the material at a generally high rate. The conduit 122 can be made from a metal, such as aluminum or copper. In other embodiments, the conduit 122 is made from conductive polymers, composites, or other metallic-based materials with thermally conductive properties. The conduit 122 can reduce the vapor temperature. As vapor travels through the conduit 122, it is in contact with the inner surface of the conduit 122 thereby reducing the temperature so it can be subsequently inhaled by the user at a proper temperature. In another embodiment, the mouth piece 124 is made substantially of glass or other substantially non-conductive material that allows the user to safely place his or her mouth on the mouth piece 124 to inhale the vapors. The conduit 122 is rotatable about a base 128 of the mouth piece 124, for positioning the conduit 122 in a first, storage-position 123, best illustrated in FIGS. 5-6 to a second, use-position 125, best illustrated in FIGS. 7-8.

Referring now again primarily to FIGS. 1-4, the assembly 100 further includes a body 130. The body 130 can encapsulate the spiral glass body 108, the heating element 114, the material placement zone 120, and the conduit 122, when the conduit 122 is in the first, storage-position 123. In an alternative embodiment, the body 130 is shaped to fit substantially within a standard-sized clothing pocket. The body 130 can include a cover 132 hingedly coupled to a sidewall of the body 130 for selectively closing and opening a top portion of the assembly 100, for selective storage, refilling, and use of the assembly 100. The cover 132 can define an aperture 134 sized and operably configured to allow at least a portion of the conduit 122 to pass therethrough, in the second, use-position 125 (FIG. 8).

The body 130 further includes walls 136 defining a cavity 138 within the body 130. The spiral glass body 108 and the heating element 114 define the heat engine of the assembly 100, or the heating assembly 107, that is disposed within the cavity 138 a distance 140 away from the walls 136. In one embodiment, the spiral glass body 108 does not contact any of the walls 136 of the body 130. Advantageously, the distance 140 defines an air gap 142 between the spiral glass body 108 and the walls 136, the air gap 142 operably configured to thermally shield portions of the body 130 from heat radiating from the heating element 114 and heat radiating from heated air flowing through the spiral airflow passage 110. In one embodiment, the walls 136 defining the cavity 138 are encapsulated within the body 130. In another embodiment, the walls 136 are comprised of a material that has low thermal conductivity in order to protect other components, such as electronic components, that may be located within another section of the body 130, from overheating. In some embodiments, the walls 136 are comprised of a non-conductive polymer material, such as polytetrafluoreoethylene (PTFE). In yet another embodiment, the air gap 142 extends substantially the entire external surface area of the spiral glass body 108 that faces the walls 136 of the cavity 138.

At least one of the walls 136 can include a thermally reflective material 137. The thermally reflective material 137 is defined herein as any material that substantially reflects heat emitted by the heat engine at normal operating temperatures of the assembly 100 for reducing heat loss through thermal radiation. In one embodiment, the thermally reflective material 137 includes a copper-nickel alloy, which exhibits high thermal reflectivity. Most of the thermal radiation emitted by objects is infrared. Accordingly, in one embodiment, the thermally reflective material 137 is operably configured to substantially reflect infrared emissions at normal operating temperatures of the assembly 100. The thermally reflective material 137 is preferably able to withstand high vapor temperatures of the assembly 100. As best illustrated in FIG. 3, thermal emissions 139 from the heating assembly 107 travel outwardly toward the walls 136, which thermal emissions 139 are subsequently reflected back towards the heating assembly 107 by the thermally reflective material 137 within the walls 136. This increases the thermal efficiency of the assembly 100 because heat loss through radiation can be conserved and redirected back towards the heating assembly 107 to remain within the cavity 138. The assembly 100 is preferably compact, discrete and easily transportable within a pant or other type of pocket, e.g., shirt, jacket, etc., of a user. Accordingly, it is particularly desirable to reduce the number of batteries required to power the assembly 100 for portable use. Increasing thermal efficiencies decreases the amount of power required to be supplied to the heating element 114 and, therefore, can reduce the number of batteries required to operate the assembly 100.

The thermally reflective material 137 can be included in the walls 136 by impregnating the walls 136 with the thermally reflective material 137. In one embodiment, the thermally reflective material 137 can be impregnated within the walls 136 by electrophoresis, which, as is known in the art, includes applying a positive and a negative charge to particles, resulting in dispersion of said particles within a material. In one embodiment, electrophoresis is used to impregnate the wall 136, which may be substantially made of a polymer material, with copper-nickel particles. Other methods can also be used to impart thermally reflective materials 137 to the walls 136. In an alternative embodiment, a separate thermally reflective layer can be applied to a surface of the walls 136 facing the cavity 138. An additional advantage of reflecting thermal energy off the walls 136 towards the heating assembly 107 is that heat can be more readily retained within the cavity 138, as opposed to radiating toward other compartments of the assembly 100, such as the areas that include electronic components.

The assembly 100 can include a temperature control feature 146 having a user-input interface 148, a digital display 150, and a temperature controller 152. The user-input interface 148 is operably configured to receive a user input for controlling a temperature within the body 130. In one embodiment, the user-input interface 148 is operably configured to receive the user input for controlling a temperature of the heating element 114. In another embodiment, the user-input interface 148 is formed as a pair of push-buttons, where a first one of the pair of push-buttons can be pushed to actuate an increase in temperature, via a first, pressure sensor mounted on the temperature controller 152; and a second one of the pair of push-buttons can be pushed to actuate a decrease in temperature, via a second, pressure sensor mounted on the temperature controller 152. In other embodiments, the user-input interface 148 can be a touchscreen, a keypad, a switch, a scroller, a mouse, or any other device capable of receiving a user's input for controlling temperature within the body 130. The digital display 150 is operably configured to display information associated with the temperature within the body 130. In one embodiment, the digital display 150 is operably configured to display information associated with a temperature detected by a temperature sensor disposed within the body 130.

The temperature controller 152 is communicatively coupled to the user-input interface 148 and the digital display 150. The temperature controller 152 can be formed as a microcontroller, a microprocessor, or other data processing device. The temperature controller 152 is operable to control a temperature output of the heating element 114 according to information received from the user-input interface 148. The temperature controller 152 is preferably disposed a sufficient distance from the heating engine so as not to result in overheating of the temperature controller 152. In a further embodiment, the temperature controller 152 is operable to control the temperature output of the heating element 114 using techniques, such pulse width modulation (PWM) techniques and control loop feedback methods, such as, for example, a proportional integral derivative controller algorithm.

A removable, rechargeable battery 154 can be provided within the body 130. In one embodiment, the battery 154 is substantially enclosed by a polymer-based covering, for convenient storage and removal. An interface 156 is provided at a sidewall of the body 130, the interface 156 operable to recharge the battery 154, while the battery 154 is within the body 130. The interface 156 can be in the form of a USB port, mini USB port, or other interface capable of receiving power to charge the battery 154. In another embodiment, the battery 154 is operably configured for recharging via a wall charger.

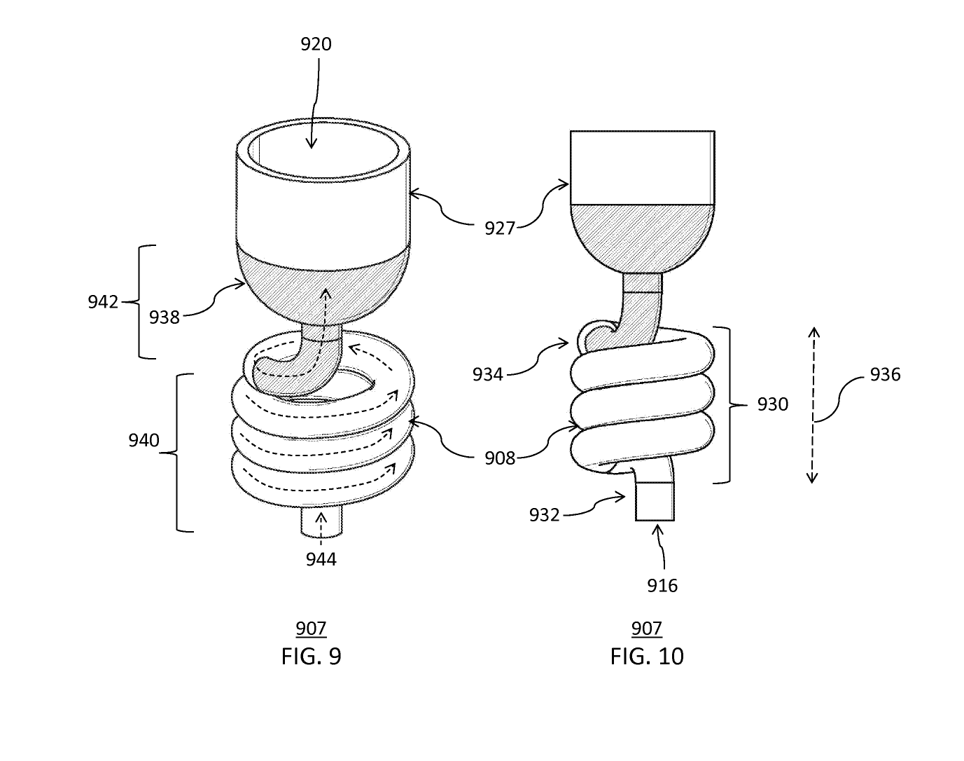

Referring now primarily to FIGS. 9-10, an alternative embodiment of a heating assembly 907, i.e. heat engine, is illustrated. Like the heating assembly 107, the heating assembly 907 includes a heating element (not shown), thermally coupled to a spiral glass body 908 and disposed within sprial windings 930 of the spiral glass body 908. The spiral glass body 908 preferably includes approximately three spiral windings 930 having a distal winding end 932 and a proximal winding end 934, opposite the distal winding end 932. The “winding end,” as used herein, is intended to indicate a portion of a body including at least one continuous spiral winding that bends away from a plane defined by the spiral winding such that the spiral motion of the at least one continuous spiral winding can be said to terminate at the winding end. In one embodiment, the winding ends 932, 934 bend substantially perpendicularly away from a plane defined by the spiral windings 930, as illustrated in FIG. 9-10. In another embodiment, the distal winding end 932 defines a distal opening 916 for receiving a stream of air 944 therethough from the external environment for the convection heating process. In some embodiments, the spiral windings 930 define a longitudinal spiral winding length 936 of approximately 16 milimeters (mm). In other embodiments, the distal winding end 932 is approximately 5 mm in length.

The proximal winding end 934 can be disposed between the spiral windings 930 and a container 927. Like the container 127, the container 927 includes a material placement zone 920, where herbal products are placed, and can provide a support surface for a receptacle that receives the herbal product therein. The container 927 can be in the form of a cup, a bowl, a tray, or other container body operably configured to hold the receptacle within a cavity or on a support surface thereof, for being heated by heated air 944 flowing through the spiral glass body 908. In one embodiment, the container 927 is made substantially of glass. In another embodiment, the proximal winding end 934 is approximately 5 mm in length. In another embodiment, the proximal winding end 934 is between approximately 5-6 mm in length.

In a preferred embodiment, a thermally conductive layer 938 covers at least a portion of an outer surface of the spiral glass body 908. In one embodiment, the thermally conductive layer covers at least a portion of the proximal winding end 934 and at least a portion of the container 927, as illustrated in FIGS. 9-10. In this embodiment, the container 927 is preferably made substantially of a glass material. The portion of the spiral glass body 908 that is not covered with the thermally conductive layer defines a pre-heating chamber 940. In one embodiment, the pre-heating chamber 940 includes the spiral windings 930 and the distal winding end 932. The pre-heating chamber 940 allows air 944 flowing into the distal opening 916 from the external environment to be pre-heated prior to being heating by a primary heating chamber 942 further downstream from the distal opening 916. The pre-heating chamber 940 advantageously reduces thermal stress to the glass material that could result from heating the glass too rapidly, potentially causing fracturing of the glass material. In one embodiment, the pre-heating chamber 940 heats air 944 flowing through it to approximately 250 degrees Fahrenheit.

The primary heating chamber 942 is operably configured to heat air 944 flowing therethrough up to a proper vaporizing temperature to vaporize the herbal product within the material placement zone 920. In one embodiment, the primary heating chamber 942 heats air 944 flowing through it to approximately 680 degrees Fahrenheit. The primary heating chamber 942 is defined by the portion of the spiral glass body 908 that is coated with the thermally conductive layer 938. The thermally conductive layer 938 is preferably a relative uniform, thin layer that provides a continuous coating on an outer surface of the spiral glass body 908. As used herein, the term “spiral glass body” is defined as a body made substantially of a glass material and, also, including a portion that is spiral shaped. In one embodiment, the primary heating chamber 942 includes the proximal winding end 934 and a bottom portion of the container 927 connected to the proximal winding end 934. In another embodiment, the thermally conductive layer 938 covers an outer surface area of the proximal winding end 934 deliminted by approximately 5 mm in length. In yet another embodiment, the thermally conductive layer 938 covers an outer surface area of the container 927 extending from an absolute bottom end of the container 927 upwardly approximately 5.42 mm towards a top rim of the container 927.

As discussed above, glass provides a clean air solution to the typical metallic tasting air resulting from vaporizers utilizing metallic heating elements that directly contact the airflow. On the other hand, glass is not generally known to be a thermally conductive material suitable for optimum heating as required with vaporizing. For example, quartz glass has a thermal conductivity of 0.8-0.93 watts per meter per Kelvin (W*m⁻¹*K⁻¹), while copper has a thermal conductivity of 385-401 watts per meter per Kelvin. In one embodiment, the thermally conductive layer 938 has a thermal conductivity of between about 600 to about 740 watts per meter per Kelvin. In an alternative embodiment, the thermally conductive layer 938 has a thermal conductivity outside of these ranges. Both the thermal conductivity of the thermally conductive layer 938 and the dimensions of the primary heating chamber 942, which are defined by the thermally conductive layer 938, are operably configured to heat air 944 flowing from the outside environment to the material placement zone 920 up to at least a required vaporization temperature of an herbal product.

The thermally conductive layer 938 covers at least a portion of the outer surface of the spiral glass body 908. Applying the thermally conductive layer 938 significantly reduces a thermal inertia of the glass body 908. As is known in the art, thermal inertia is the degree of slowness with which the temperature of a body approaches that of its surroundings, and which is partly dependent on its thermal conductivity. Without the thermally conductive layer 938, the spiral glass body 908 is much more slowly heated, which increases a time period required to sufficiently heat the herbal product for convection-based vaporizing and increases power consumed by heating the heating element, each of which contributes to thermal inefficiency. The thermally conductive layer 938 advantageously provides a flexible method of controlling, concentrating, and restricting heat to the area where it is most efficiently utilized, i.e. the primary heating chamber 942. In other words, it creates a type of thermal battery that collects, stores, and releases heat in an efficient manner.

In one embodiment, the thermally conductive layer 938 includes Titanium CarboNitride, Chromium Nitride, Aluminum Titanium Nitride, Aluminum Titanium Chronium Nitride, and/or Zirconium Nitride, each of which exhibits high thermal conductivity. In a preferred embodiment, the thermally conductive layer 938 includes boron. In another preferred embodiment, the thermally conductive layer 938 includes boron nitride, which exhibits excellent thermal stability. In yet another embodiment, the thermally conductive layer 938 includes a boron nitride powder mixed with a thermally conductive adhesive. The adhesive allows the boron nitride powder to adhere to the spiral glass body 908. In another embodiment, the thermally conductive layer 938 covers less than about one-third of the outer surface of the spiral glass body 908. In a further embodiment, the thermally conductive layer 938 covers the proximal winding end 934, and not the distal winding end 932. In yet a further embodiment, the thermally conductive layer 938 covers the proximal winding end 934, and neither the distal winding end 932 nor a substantial portion of the sprial windings 930. In another embodiment, the thermally conductive layer 938 covers a portion of an outer surface of the spiral glass body 908 and a portion of an outer surface of the container 927.

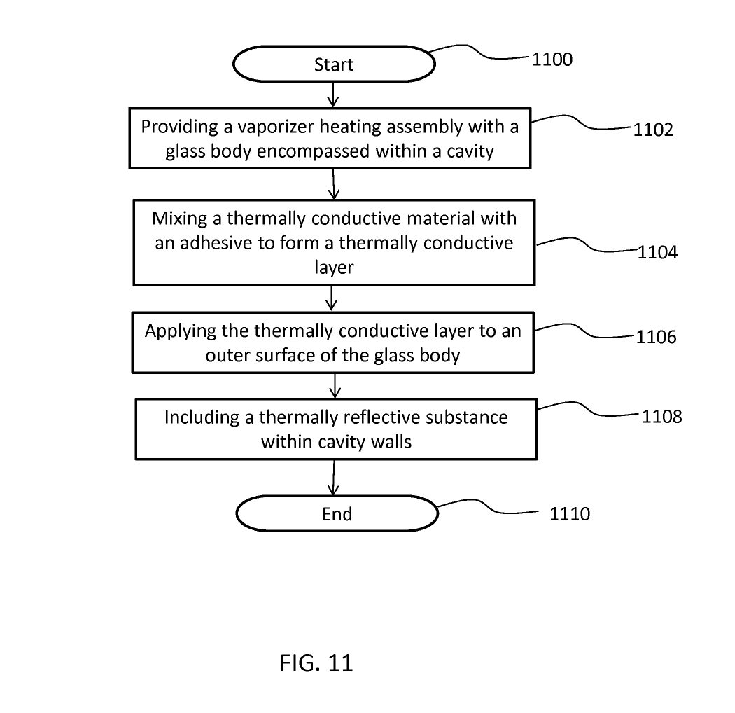

Referring now primarily to FIG. 11 in conjunction with FIGS. 9-10, a flow chart illustrating an exemplary method of improving a thermal efficiency of a vaporizer heating assembly having a glass airflow passage is presented. The method begins with step 1100 and immediately proceeds to step 1102, where the vaporizer heating assembly is provided. In one embodiment, the vaporizer heating assembly is a portable vaporizer heating assembly operable to fit within a standard-sized pant pocket and also resembling a standard-sized cellular phone. The portable vaporizer heating assembly includes a spiral glass body defining a spiral airflow passage, a heating element thermally coupled to the spiral glass body, and a material placement zone downstream from the spiral airflow passage. In another embodiment, the spiral glass body and the heating element define a heat engine of the vaporizer heating assembly and the heat engine is disposed within a cavity defined by a plurality of walls. The plurality of walls can be said to substantially encapsulate the heat engine.

In step 1104, a thermally conductive material is mixed with an adhesive that is also thermally conductive, forming a thermally conductive layer. In one embodiment, a boron nitride powder is mixed with an adhesive to form a paste-like substance. The adhesive is operably configured to allow the paste-like substance to adhere to an outer surface of the spiral glass body, even under a normal operating temperature of the vaporizer heating assembly. The adhesive is preferably able to withstand high vapor temperatures over an estimated lifetime of the vaporizer heating assembly. Selecting an adhesive that loses adhesion under such operating temperatures is not desirable. As used herein, the term “paste-like substance” is defined as a moist substance produced by mixing a dry ingredient with a liquid.

In step 1106, the thermally conductive layer is applied to at least a portion of an outer surface of the spiral glass body. In one embodiment, a boron nitride layer is applied to the portion of the outer surface of the spiral glass body. In another embodiment, the paste-like substance is applied to the portion of the outer surface of the spiral glass body. In step 1108, a thermally reflective substance is included within the plurality of walls to reflect heat. In one embodiment, the plurality of walls is operably configured to reflect infrared emissions within the cavity. In one embodiment, the thermally reflective substance includes copper-nickel. In some embodiments, the thermally reflective substance is included by impregnating the substance within the plurality of walls. In other embodiments, the thermally reflective substance is included by infusing the thermally reflective substance within the plurality of walls. Prior to infusion or impregnation, the plurality of walls can be made substantially of a polymer material, such as Teflon®. In one embodiment, the plurality of walls is impregnated with copper-nickel. In yet other embodiments, the thermally reflective substance is included within the plurality of walls by applying a coating of the thermally reflective substance to the outer surface of the plurality of walls.

A portable hand-held vaporizer has been disclosed that provides a body in the shape that is able to fit within a standard-sized pant pocket and also resembling a standard-sized cellular phone. This novel vaporizer utilizes the more effective and efficient convection-based heating while still maintaining a compact design. To achieve this, the vaporizer discloses an airflow chamber directing outside ambient air through a heating element into an herb placement zone, which is located in the near proximity to a distal end of a conduit where it is subsequently inhaled by a user. When the vapor reaches the user, the temperature has reduced to a range that is safe and comfortable for the user. The vaporizer further provides that the conduit may be advantageously placed in a first and second position that allows the purpose and nature of the device to be relatively unidentifiable to the viewing public. Additionally, the airflow chamber is designed with materials that optimize thermal efficiency and create the purest and most healthful vapor results available, cleanly through a sealed non-contaminated glass air passage such that air flowing through the air passage does not contact a metallic heating element. Moreover, the compact spiral design of the airflow chamber and the heating element provides thermal efficiency important for power requirements and size restrictions of the portable hand-held vaporizer. A thermally conductive layer, preferably including boron nitride, is applied to the glass air passage to overcome the low thermal conductivity of glass by efficiently directing heat to an optimal location along the glass air passage. And, a thermally reflective substance is included in walls encapsulating the heating engine of the assembly, so as to conserve heat by reflecting radiating heat back towards the heating engine.

What is claimed is:

1. A vaporizer heating assembly comprising:
a glass body defining an airflow passage;
a heating element thermally coupled to the glass body;
a material placement zone downstream from the airflow passage; and
a thermally conductive layer covering at least a portion of an outer surface of the glass body.

2. The vaporizer heating assembly in accordance with claim 1, wherein:
the thermally conductive layer comprises boron.

3. The vaporizer heating assembly in accordance with claim 1, wherein:
the thermally conductive layer comprises a boron nitride powder and an adhesive.

4. The vaporizer heating assembly in accordance with claim 1, wherein:
the thermally conductive layer covers less than about one-third of the outer surface of the glass body.

5. The vaporizer heating assembly in accordance with claim 1, wherein:
the glass body includes at least one spiral winding between a distal winding end and a proximal winding end, opposite the distal winding end; and
the thermally conductive layer covers the proximal winding end and not the distal winding end.

6. The vaporizer heating assembly in accordance with claim 1, wherein:
the glass body comprises:
a spiral glass body defining a spiral airflow passage, and
a container fluidly coupled to the spiral airflow passage, the container including the material placement zone; and
the thermally conductive layer covers a portion of an outer surface of the spiral glass body and a portion of the container.

7. The vaporizer heating assembly in accordance with claim 1, further comprising:
a body including a plurality of walls defining a cavity within the body, the glass body and the heating element disposed within the cavity, and the plurality of walls including a thermally reflective substance operably configured to reflect infrared emissions within the cavity.

8. A vaporizer heating assembly comprising:
an airflow passage thermally coupled to a heating element;
a material placement zone downstream from the airflow passage; and
a body including a plurality of walls defining a cavity within the body, the airflow passage and the heating element disposed within the cavity, and at least one of the plurality of walls including a thermally reflective material.

9. The vaporizer heating assembly in accordance with claim 8, wherein:
the thermally reflective material comprises copper-nickel.

10. The vaporizer heating assembly in accordance with claim 8, wherein:
the at least one of the plurality of walls is comprised of a polymer material impregnated with copper-nickel.

11. The vaporizer heating assembly in accordance with claim 8, wherein:
the thermally reflective material is operably configured to reflect infrared emissions within the cavity.

12. The vaporizer heating assembly in accordance with claim 8, wherein:
the airflow passage is defined by a glass body.

13. The vaporizer heating assembly in accordance with claim 8, wherein:
the airflow passage is formed as a spiral airflow passage defined by a spiral glass body.

14. The vaporizer heating assembly in accordance with claim 8, further comprising:
a thermally conductive layer covering at least a portion of an outer surface of a glass body defining the airflow passage.

15. A method of improving a thermal efficiency of a vaporizer heating assembly having a glass airflow passage, the method comprising:
providing a vaporizer heating assembly including:
a glass body defining an airflow passage,
a heating element thermally coupled to the glass body, and
a material placement zone downstream from the airflow passage; and
applying a thermally conductive layer to at least a portion of an outer surface of the glass body.

16. The method in accordance with claim 15, further comprising:
applying a boron nitride layer to at least a portion of an outer surface of the glass body.

17. The method in accordance with claim 15, further comprising:
mixing a boron nitride powder with an adhesive to form a paste-like substance; and
applying the paste-like substance to at least a portion of an outer surface of the glass body.

18. The method in accordance with claim 15, further comprising:
providing the glass body and the heating element within a cavity defined by a plurality of walls; and
including a thermally reflective substance within the plurality of walls to reflect infrared emissions within the cavity.

19. The method in accordance with claim 15, further comprising:
providing the glass body and the heating element within a cavity defined by a plurality of walls; and
including copper-nickel within the plurality of walls to reflect infrared emissions within the cavity.

20. The method in accordance with claim 15, further comprising:
providing the glass body and the heating element within a cavity defined by a plurality of walls; and
impregnating at least one of the plurality of walls with copper-nickel.
